(19) United States
(12) Patent Application Publication
(10) Pub. No.: US 2010/0142933 A1
(43) Pub. Date: Jun. 10, 2010

(54) FOG GENERATOR

(75) Inventor: Alfons Vandoninck, Opglabbeck (BE)

Correspondence Address: DARBY & DARBY P.C., P.O. BOX 770, Church Street Station, New York, NY, 10008-0770, US

(73) Assignee: Bandit NV, Opglabbeck (BE)

(21) Appl. No.: 12/596,028

(22) PCT Filed: Apr. 23, 2008
(86) PCT No.: PCT/EP2008/054930

§ 371 Date: Dec. 4, 2009

(30) Foreign Application Priority Data
Apr. 27, 2007 (EP) 07008600.4

(51) Int. Cl. A61L 9/03

(52) U.S. Cl. 392/386

(57) ABSTRACT

The present invention is directed to a fog generator comprising a vessel that contains a fog generating fluid, driving means for driving the fluid from the vessel into a heat exchanger which transforms the fog generating fluid into steam and is connected with the vessel, a means for ejecting the steam in the form of a fog and connected to the heat exchanger, a valve for switching on and off the fluid flow from the vessel into the heat exchanger, and a means for purging non-ejected steam out of the heat exchanger into the ambient connected to the means for driving the fluid, characterized in that the means for driving the fluid and the means for purging are connected such that by switching off the fluid flow non-ejected steam can be purged out of the heat exchanger

### FIELD OF THE INVENTION

The present invention relates to a device for generating fog.

### BACKGROUND OF THE INVENTION

Fog generators are used in a variety of applications. They can be used in applications concerning security, e.g. for generating a fog screen by which goods or valuables are screened out from the intruder's sight, or for simulating fire as a training aid for emergency services or security forces. They can also be used in applications concerning entertainment, e.g. for creating lighting effects on stage, etc.

According to the state of the art, a main working principle of a fog generator is as follows: a fog generating fluid is driven into a heat exchanger by a pump or a propellant gas; in the heat exchanger, the fog generating fluid is heated and transferred to steam; the steam ejects then at the end of the heat exchanger in the form of a fog into the ambient.

An important problem associated with current executions is that, upon switching off the fog generating fluid flow, the heat exchanger is cut off from pump pressure or propellant gas pressure. The pressure in the heat exchanger drops to atmospheric ambient pressure. Consequently, an amount of fog generating fluid is not driven out and remains in the heat exchanger. The high temperature in the heat exchanger and incoming oxygen causes decomposition of the fog generating fluid remaining inside the heat exchanger. Some components resulting from this decomposition are very corrosive and damage the inside of the heat exchanger.

In EP1402225, the above problem is described and a fog generator alleviating it is proposed. A propellant gas selected from the group of halogenated hydrocarbons is used to drive the fog generating fluid into the heat exchanger. When the fog generating fluid flow from the vessel into the heat exchanger is cut off, the propellant gas dissolved in the fog generating fluid which remained in the heat exchanger expands and forces the fluid to flow towards the heat exchanger exhaust.

However, due to severe legal restrictions on the use of greenhouse gases, a preferred fog generator would be a fog generator having an alternative way to remove non-ejected steam from the heat exchanger.

Another fog generator is described in GB 640 266 A, wherein by means of a first circuit a fog fluid is driven via a nozzle into a heat exchanger. Via a second and separated circuit, a jet of carbon dioxide or nitrogen is provided via another nozzle in close proximity to the fluid delivering nozzle to atomize and propel the fluid into the heat exchanger. When using only the nozzle providing carbon dioxide or nitrogen, the heat exchanger may be purged.

GB-A-1 039 729 describes a fog generator wherein the fog generating fluid is driven to the heat exchanger by means of carbon dioxide propellant gas. A valve switches on and off the propellant gas flow to force the fog generating fluid into the heat exchanger. As soon as the fluid level in the fluid containing vessel has dropped low enough, the fog generating fluid will cease to flow and only carbon dioxide will flow into the heat exchanger thereby purging the heat exchanger. A problem of this apparatus is that the heat-exchanger can only be purged after the fluid level in the vessel dropped low enough. Whether it is possible to purge the heat exchanger or not, depends on the fog fluid level.

Although not disclosed in the prior art, another problem associated with current fog generators is the appearance of malodors after using it. These malodors are irritating. It is therefore clear that a preferred fog generator would be a fog generator which does not generate malodors upon usage.

In contrast to state-of-the-art fog generators, a fog generator in accordance to the present invention is able to purge the heat exchanger independently of fog fluid level in the fog fluid containing vessel. Further, it does not make use of greenhouse gases and it also does not generate malodors upon usage.

### SUMMARY OF THE INVENTION

The present invention is directed to a fog generator comprising a vessel that contains a fog generating fluid, driving means for driving the fluid from the vessel into a heat exchanger which transforms the fog generating fluid into steam and is connected with the vessel, a means for ejecting the steam in the form of a fog and connected to the heat exchanger, a valve for switching on and off the fluid flow from the vessel into the heat exchanger, and a means for purging non-ejected steam out of the heat exchanger into the ambient connected to the means for driving the fluid, characterized in that the means for driving the fluid and the means for purging are connected such that by switching off the fluid flow non-ejected steam can be purged out of the heat exchanger.

### BRIEF DESCRIPTION OF THE DRAWINGS

FIG. 1 shows an embodiment of a fog generator in accordance with the present invention.

FIG. 2 shows another embodiment of a fog generator in accordance with the present invention.

### DESCRIPTION OF THE INVENTION

A person skilled in the art will understood that the embodiments described below are merely illustrative in accordance with the present invention and not limiting the intended scope of the invention. Other embodiments may also be considered.

The present invention provides a fog generator comprising a vessel that contains a fog generating fluid, driving means for driving the fluid from the vessel into a heat exchanger which transforms the fog generating fluid into steam and is connected with the vessel, a means for ejecting the steam in the form of a fog and connected to the heat exchanger, a valve for switching on and off the fluid flow from the vessel into the heat exchanger, and a means for purging non-ejected steam out of the heat exchanger into the ambient connected to the means for driving the fluid, characterized in that the means for driving the fluid and the means for purging are connected such that by switching off the fluid flow non-ejected steam can be purged out of the heat exchanger.

By connecting the means for driving the fluid and the means for purging such that by switching off the fluid flow non-ejected steam can be purged out of the heat exchanger, the heat exchanger can be purged independently of fog fluid level in the fog fluid containing vessel. Consequently, after each fog generating period, the heat exchanger can be purged.

Further, it has surprisingly been found that a fog generator in accordance with the invention does not generate malodors upon usage. Without being bound by any theory, it is now believed that the problem of decomposing non-ejected steam leads to the problem of these malodors. In particular oxidation results in formation of amongst others carbon dioxide, carbon monoxide and aldehydes. Especially formaldehyde and acetaldehyde are unacceptable, because of their toxicity and irritating odor. When using a means for purging, the non-ejected steam is driven out of the heat exchanger before decomposition may occur.

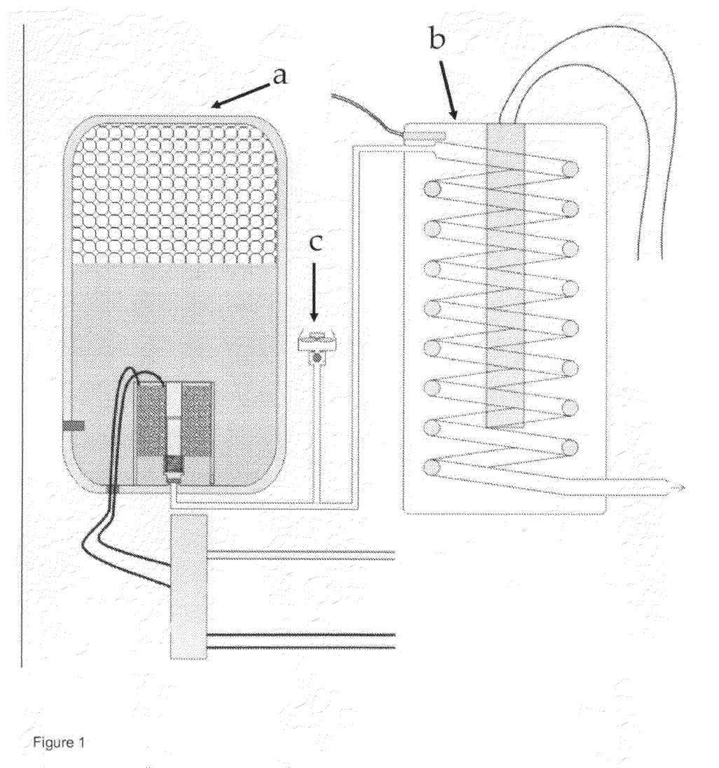

In an embodiment in accordance with the present invention, the means for purging non-ejected steam out of the heat exchanger may be a gas purging unit, which may be any device suitable for purging a heat exchanger channel, such as but not limited to an air pump, a fan, a blower, a compressor or a vessel containing a compressed gas. Preferably, the blowing unit is connected at the inlet of the heat exchanger channel or channels and forces the non-ejected steam to flow towards the outlet. In FIG. 1, a fog generator in accordance with this embodiment is shown comprising a vessel (a) containing a fog fluid and connected to a heat exchanger (b), and a gas purging unit (c) providing purge gas to the heat exchanger.

The purging unit provides purge gas to the heat exchanger. Purge gas may be any gas which is low toxic, low inflammable and low corrosive, such as but not limited to ambient air or nitrogen. Preferably, ambient air is used.

An air pump, a fan, a blower or a compressor may, in the context of the present invention, be used for providing purge gas, such as but not limited to ambient air for quickly removing non-ejected steam from the heat exchanger channel and replace it with purge gas before decomposition of the fog generating fluid occurs. A vessel containing a compressed gas, such as compressed nitrogen, and accompanied with a valve for opening and closing the vessel may be also suitable for quickly purging the heat exchanger channel.

Further, the purging unit may be accompanied by a check valve. Check valves are self-activating one directional valves that permit gases and liquids to flow in only one direction, preventing process flow from reversing. Purge gas is allowed to flow through the valve in the desired direction, while fog generating fluid backflow forces the valve closed. In the context of the present invention, the open direction of the check valve is from the purge gas bulk or the ambient air towards the heat exchanger inlet. The closed direction is from the heat exchanger inlet towards the purge gas bulk or ambient air, thereby preventing the fog generating fluid, which during a fog generation period flows from vessel to heat exchanger, from entering the purge gas bulk or the ambient air.

After the fog generator stops ejecting fog, the purge gas may be provided in a continuous gas flow during a period of up to about 15 minutes, and preferably about 10 minutes. Or the purge gas may also be provided with a number of short gas pulses, preferably 3 pulses per minute during about 10 minutes.

In another embodiment in accordance with the present invention, the vessel containing the fog generating fluid further contains a propellant gas, the driving means for driving the fog generating fluid into the heat exchanger is the propellant gas and the means for purging comprise a connection from the propellant gas volume of the vessel to the heat exchanger for purging the heat exchanger with propellant gas. So, in this case the vessel contains propellant gas both for driving the fog generating fluid from the vessel into the heat exchanger and for purging the heat exchanger.

The propellant gas may be any low toxic, low inflammable and environmentally acceptable gas. Preferably, it may be an inert gas, such as but not limited to nitrogen, or a noble gas, such as but not limited to helium, neon, or argon. It may also be a mixture of noble gasses or a mixture of inert and noble gasses, such as but not limited to a mixture of argon and nitrogen.

The connection from the propellant gas volume in the vessel to the heat exchanger may comprise a valve for controlling the propellant gas purge flow. The valve may be controlled by a valve controller and may be any valve suitable for controlling a gas flow.

After the fog generator stops ejecting fog, the propellant gas may be provided via the connection to the heat exchanger in a continuous gas flow during a period of up to about 15 minutes, and preferably about 10 minutes. Or the propellant gas may also be provided with a number of short gas pulses.

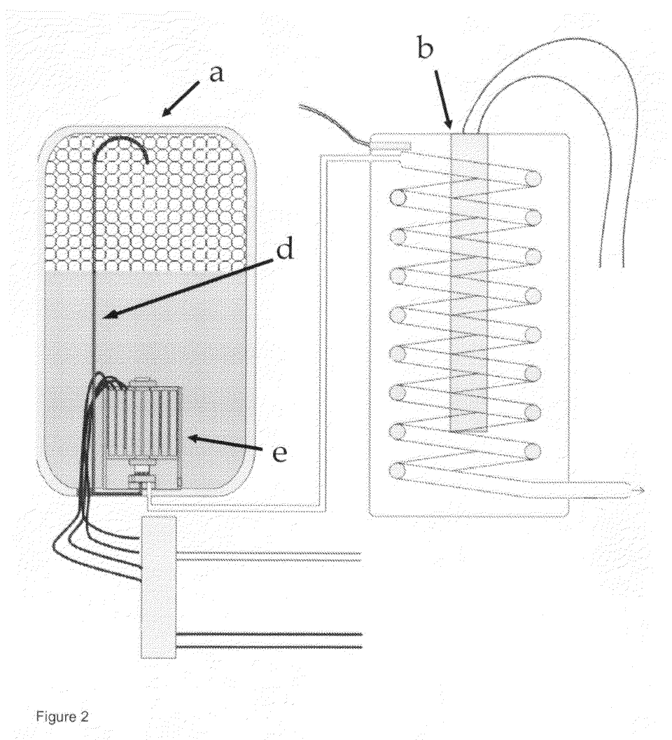

In a preferred embodiment in accordance with the present invention, the valve for controlling the propellant gas purge flow is suitable for switching between connecting the fog generating fluid volume of the vessel with the heat exchanger or connecting the propellant gas volume of the vessel with the heat exchanger. The purge gas flow from the propellant gas volume of the vessel is controlled by the same valve which controls the fog generating fluid flow from the fog generating fluid volume of the vessel to the heat exchanger. In other words, during a fog generation period, the valve passes fog generating fluid to the heat exchanger, while as soon as the fog generation period stops, the valve passes propellant gas to the heat exchanger, thereby removing remaining non-ejected steam from the heat exchanger. This valve may be any valve suitable for switching between two connections, such as but not limited to a 3-directional valve or a disc valve with a stepper motor. In FIG. 2, a fog generator in accordance with this embodiment is shown comprising a vessel (a) containing fog fluid and a propellant gas, and connected to a heat exchanger (b). The propellant gas is via a connection (d) and a disc valve (e) transported to the heat exchanger.

By using propellant gas as purge gas, there is no need for a purging unit and a check valve, resulting in a simplified and less expensive construction. However, extra volume propellant gas may be provided, leading to the need for a higher vessel pressure (and optionally a more robust vessel) or a larger vessel. Preferably, a higher vessel pressure is used, which generates about 30 liter expanded propellant gas, when using a vessel of 0.45 liter propellant gas volume at 180 bars instead of 110 bars.

1. A fog generator comprising a vessel that contains a fog generating fluid, driving means for driving the fluid from the vessel into a heat exchanger which transforms the fog generating fluid into steam and is connected with the vessel, a means for ejecting the steam in the form of a fog and connected to the heat exchanger, a valve for switching on and off the fluid flow from the vessel into the heat exchanger, and a means for purging non-ejected steam out of the heat exchanger into the ambient connected to the means for driving the fluid, characterized in that the means for driving the fluid and the means for purging are connected such that by switching off the fluid flow non-ejected steam can be purged out of the heat exchanger.

2. A fog generator according to claim 1, wherein the means for purging comprise a purging unit suitable for purging a heat exchanger channel, such as but not limited to an air pump, a fan, a blower, a compressor or a vessel containing compressed gas.

3. A fog generator according to claim 2, wherein the purging unit purges the heat exchanger with ambient air.

4. A fog generator according to claim 1, wherein the vessel containing the fog generating fluid further contains a propellant gas, wherein the driving means is the propellant gas and wherein the means for purging comprise a connection from the propellant gas volume of the vessel to the heat exchanger for purging the heat exchanger with propellant gas.

5. A fog generator according to claim 4, wherein the connection comprises a valve for controlling the propellant gas purge flow.

6. A fog generator according to claim 5, wherein the valve is suitable for switching between connecting the fog generating fluid volume of the vessel with the heat exchanger and connecting the propellant gas volume of the vessel with the heat exchanger.
